(19) United States
(12) Patent Application Publication
(10) Pub. No.: US 2005/0137264 A1
(43) Pub. Date: Jun. 23, 2005

(54) Modafinil compositions

(76) Inventors: Ashish Anilbhai Patel, Kendall Park, NJ (US); Gary Barbera, Medford, NJ (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/743,366

(22) Filed: Dec. 22, 2003

(51) Int. Cl. A61K 9/14; A61K 31/165

(52) U.S. Cl. 514/618; 424/489

(57) ABSTRACT

A pharmaceutical composition comprising modafinil in the form of particles, wherein greater than 5% to about 50% of the cumulative total of the modafinil particles in the composition have a particle size greater than about 200 microns as determined by a U.S. Sieve No. 75. The present inventors have unexpectedly determined that the pharmaceutical compositions of the invention exhibit comparable stability, dissolution, and bioavailability as compared to Provigil® which is a commercially-available modafinil formulation.

### FIELD OF THE INVENTION

The present invention provides a pharmaceutical composition comprising modafinil in the form of particles, wherein greater than 5% to about 50% of the cumulative total of the modafinil particles in the composition have a particle size greater than about 200 microns.

### BACKGROUND OF THE INVENTION

Modafinil (C₁₅H₁₅NO₂S), also known as 2-(benzhydrylsulfinyl) acetamide, or 2-[(diphenylmethyl)sulfinyl]acetamide, is a synthetic acetamide derivative with wake-promoting activity, the structure of which has been described in French Patent No. 78 05 510 and in U.S. Pat. No. 4,177,290 and which has been approved by the United States Food and Drug Administration for use in the treatment of excessive daytime sleepiness associated with narcolepsy. Modafinil is commercially available under the trademark Provigil® from Cephalon, Inc. Provigil® is supplied as tablets containing 100 mg or 200 mg modafinil, with lactose, corn starch, magnesium silicate, croscarmellose sodium, povidone, magnesium stearate and talc.

U.S. Pat. No. 4,927,855 describes the levorotatory isomer of benzhydrylsulfinylacetamide and its use as an antidepressant and a stimulant in the treatment of hypersomnia and Alzheimer's Disease.

U.S. Pat. No. 5,618,845 describes a pharmaceutical composition comprising substantially homogeneous mixture of modafinil particles wherein at least about 95% of the cumulative total of modafinil particles in the composition have a diameter of less than about 200 microns.

### SUMMARY OF THE INVENTION

The invention provides a pharmaceutical composition comprising modafinil in the form of particles, wherein greater than 5% to about 50% of the cumulative total of the modafinil particles in the composition have a particle size greater than about 200 microns as determined by a U.S. Sieve No. 75.

According to another aspect, the invention provides a pharmaceutical composition comprising modafinil in the form of particles, wherein greater than 5% to about 50% of the cumulative total of the modafinil particles in the composition have a particle size greater than about 200 microns as determined by a U.S. Sieve No. 75, and less than about 8% of the cumulative total of the modafinil particles in the composition have a particle size greater than about 250 microns as determined by a U.S. Sieve No. 60.

According to another aspect, the invention provides a pharmaceutical composition comprising modafinil and calcium silicate.

According to another aspect, the invention provides a pharmaceutical composition comprising modafinil and calcium silicate in the form of particles, wherein greater than 5% to about 50% of the cumulative total of the modafinil particles in the composition have a particle size greater than about 200 microns as determined by a U.S. Sieve No. 75.

According to another aspect, the invention provides a process for preparing a pharmaceutical composition comprising modafinil and at least one silicate, said process comprising: (i) mixing modafinil and at least one silicate to form a mixture; and (ii) optionally mixing other excipients with the mixture formed in Step (i) to form a composition.

The present inventors have unexpectedly determined that the pharmaceutical compositions of the invention exhibit comparable stability, dissolution and bioavailability as compared to Provigil®, which is a commercially available modafinil composition in the form of a tablet containing 200 mg of modafinil, lactose, corn starch, magnesium silicate, croscarmellose sodium, povidone, magnesium stearate and talc.

### BRIEF DESCRIPTION OF THE DRAWINGS

FIG. 1 is a dissolution profile comparison of two modafinil compositions prepared according to the invention as compared to Provigil®.

### DETAILED DESCRIPTION OF THE INVENTION

The invention provides a pharmaceutical composition comprising modafinil in the form of particles, wherein greater than 5% to about 50% of the cumulative total of the modafinil particles in the composition have a particle size greater than about 200 microns as determined by a U.S. Sieve No. 75. Preferably from about 8% to about 30%, more preferably from about 8% to about 10%, of the cumulative total of the modafinil particles in the composition have a particle size greater than about 200 microns as determined by a U.S. Sieve No. 75.

In one embodiment of the invention, less than about 8% of the cumulative total of the modafinil particles in the composition have a particle size greater than about 250 microns as determined by a U.S. Sieve No. 60.

In one embodiment of the invention, less than about 55% of the cumulative total of the modafinil particles in the composition have a particle size greater than about 90 microns as determined by a U.S. Sieve No. 170.

As used herein, “modafinil” refers to modafinil, its racemic mixtures, individual enantiomers, acid addition salts, such as a metabolic acid of modafinil, benzhydrylsulfinylacetic acids, and its sulfone forms, hydroxylated forms, polymorphic forms, analogs, derivatives, cogeners and prodrugs thereof. Prodrugs are known in the art as compounds that are converted to modafinil in the body of a subject.

Modafinil is present in the pharmaceutical compositions in an amount of from about 1 weight percent (wt. %) to about 99 wt. %, based on the total weight of the pharmaceutical composition. Preferably, modafinil is present in an amount of from about 30 wt. % to about 50 wt. %, more preferably about 40 wt. %, based on the total weight of the composition.

In one embodiment of the invention, a pharmaceutical composition is provided which comprises modafinil and at least one silicate. Preferred silicates include calcium silicate, sodium silicate, magnesium silicate and magnesium trisilicate. A combination of silicates may also be used. More preferably, the silicate is calcium silicate. Most preferably, the silicate is a combination of calcium silicate and magnesium trisilicate.

The silicate(s) are present in the pharmaceutical compositions in an amount of from about 0.1 wt. % to about 50 wt. %, based on the total weight of the pharmaceutical composition. Preferably, the silicate(s) are present in an amount of from about 1 wt. % to about 10 wt. %, more preferably from about 5 wt. % to about 6 wt. %, based on the total weight of the composition.

It is within the scope of the invention to prepare the pharmaceutical compositions of the invention that are “essentially free” of magnesium silicate. As used herein, “essentially free” means that the compositions contain less than 5 wt. % of magnesium silicate, based on the total weight of the composition. Preferably, the compositions contain less than 3 wt. %, more preferably less than 1 wt. % of magnesium silicate.

The pharmaceutical compositions may contain one or more excipients. Examples of excipients include, but are not limited to, diluents, disintegrants, lubricants, glidants, binders, fillers, emulsifiers, electrolytes, wetting agents, solubilizers, surfactants, colors, pigments and anti-caking agents. A combination of excipients may also be used. Preferably, the excipients meet the standards of the National Formulary (“NF”) or United States Pharmacopoeia (“USP”).

Examples of glidants include, but are not limited to, silica, magnesium trisilicate, powdered cellulose, starch, talc and tribasic calcium phosphate. A preferred glidant is silicon dioxide.

Examples of fillers or diluents include, but are not limited to, spray-dried or anhydrous lactose; sucrose; dextrose; starch; pre-gelatinized starch; polyols, such as mannitol, sorbitol and xylitol; cellulose, such as microcrystalline cellulose; and inorganic salts, such as dibasic calcium phosphate, tribasic calcium phosphate and calcium sulfate. Preferably, the filler or diluent is lactose monohydrate.

Examples of disintegrants include, but are not limited to, starch and starch derivatives, including cross-linked sodium salt of a carboxymethyl ether of starch, such as sodium starch glycolate; pre-gelatinized starch, such as Starch 1500; sodium starch glycolate; cross-linked sodium carboxymethyl cellulose, such as croscarmellose sodium; cross-linked polyvinylpyrrolidone, such as crospovidone; and microcrystalline cellulose. A preferred disintegrant is cross-linked sodium carboxymethyl cellulose.

Examples of binders include, but are not limited to, cellulose derivatives, such as microcrystalline cellulose, methylcellulose, carboxymethycellulose sodium, hydroxypropyl methylcellulose, hydroxyethyl cellulose and hydroxypropyl cellulose; polyvidone; polyvinyl pyrrolidone; gelatin; natural gums, such as acacia, tragacanth, guar and pectin; starch paste; pre-gelatinized starch; sucrose; corn syrup; polyethylene glycols and sodium alginate; ammonium calcium alginate; magnesium aluminum silicate; and polyethylene glycols. A preferred binder is pre-gelatinized starch.

Several co-processed filler-binders are commercially-available, including cellactose (α-lactose monohydrate and powdered cellulose 75:25), microcelac (α-lactose monohydrate and powdered cellulose 75:25), ludipress (93% α-lactose monohydrate, 3.5% polyvinylpyrrolidone and 3.5% crospovidone) and pharmatose DCL 40 (95% β-lactose and 5% lactitol).

Examples of lubricants include, but are not limited to, vegetable oils, such as hydrogenated vegetable oil or hydrogenated castor oil; polyethylene glycols, such as PEG-4000 and PEG-6000; stearic acid; salts of stearic acid, such as magnesium stearate, sodium stearate and sodium stearyl fumarate; mineral salts, such as talc; inorganic salts; organic salts, such as sodium benzoate, sodium acetate and sodium oleate; and polyvinyl alcohols. A preferred lubricant is magnesium stearate.

In a preferred embodiment of the invention, the pharmaceutical composition is prepared with the following excipients: a diluent selected from starch, lactose and microcrystalline cellulose; a disintegrant selected from pre-gelatinized starch, a cross-linked sodium carboxymethyl cellulose and combinations thereof; and magnesium stearate as the lubricant.

In one embodiment of the invention, the pharmaceutical compositions are prepared by a dry blending process comprising: (i) mixing modafinil and at least one silicate to form a mixture; and (ii) optionally mixing other excipients with the mixture formed in Step (i) to form a composition. In the first step, modafinil and a silicate are blended with excipients other than a lubricant. Preferably, modafinil and a silicate are thoroughly dry blended with at least one glidant, diluent, binder and disintegrant, to form a uniform dry mixture. Blenders appropriate for large scale dry blending include twin shell blenders, double cone blenders, V-blenders or bin-blenders. A preferred blender is a V-shell PK blender. High-speed, high-shear mixers may also be used. The dry mixture may also be granulated, milled into a fine powder or passed through a mesh screen. In the next step, a lubricant is blended with the dry mixture to give particles. In certain embodiments, a V-blender or bin-blenders are used. A preferred blender is a V-shell PK blender.

The pharmaceutical compositions of the invention are prepared as solid dosage forms. Solid dosage forms include capsules, caplets, powders and tablets. In one embodiment, the compositions are compressed into a tablet. Tablets may include sugar-coated tablets, film-coated tablets, enteric-coated tablets, multiple-compressed tablets, bi-layer tablets and effervescent tablets. The tablet formulation can be prepared by wet granulation, dry granulation, direct compression or by any other technique known in the pharmaceutical art.

In another embodiment, the compositions are enclosed in a capsule, preferably a hard gelatin capsule. Hard gelatin capsules are commercially-available. The hard capsules can be prepared by filling the longer end of the capsule with the composition, and slipping a cap over the top using mG2, Zanasi, or Hofliger and Karg (H&K) machines.

The pharmaceutical compositions of the invention are especially useful in the treatment of sleepiness, promotion of wakefulness, treatment of Parkinson's disease, cerebral ischemia, stroke, sleep apneas, eating disorders, stimulation of appetite and weight gain, treatment of attention deficit hyperactivity disorder and fatigue and improvement of cognitive dysfunction.

The following non-limiting examples illustrate further aspects of the invention.

### EXAMPLE 1

### Preparation of Two Modafinil Compositions Containing 200 mg of Modafinil

| | Amount per Tablet | |
|---|---|---|
| Ingredient | Composition 1A | Composition 1B |
| Modafinil | 200 mg | 200 mg |
| Calcium Silicate USP | 25.0 mg | 25.0 mg |
| Magnesium Trisilicate USP | 0.0 mg | 5.0 mg |
| Lactose Monohydrate NF | 217.5 mg | 212.5 mg |
| Pregelatinized Starch NF | 25.0 mg | 25.0 mg |
| Croscarmellose Sodium NF | 25.0 mg | 25.0 mg |
| Magnesium Stearate NF | 7.5 mg | 7.5 mg |

Modafinil Compositions 1A and 1B were prepared by a dry blending process as follows: modafinil, calcium silicate and magnesium trisilicate in the case of Composition 1B, were mixed using a V-shell PK blender to form a uniform dry mixture. Lactose, pre-gelatinized starch and croscarmellose sodium were added to the mixture and were mixed using a V-shell PK blender. Magnesium stearate was added to the mixture and mixed using a V-shell PK blender to form particles. The particles were compressed into tablets.

### EXAMPLE 2

### Dissolution Profile Comparison of Modafinil Compositions 1A and 1B Prepared in Example 1, as Compared to Provigil®

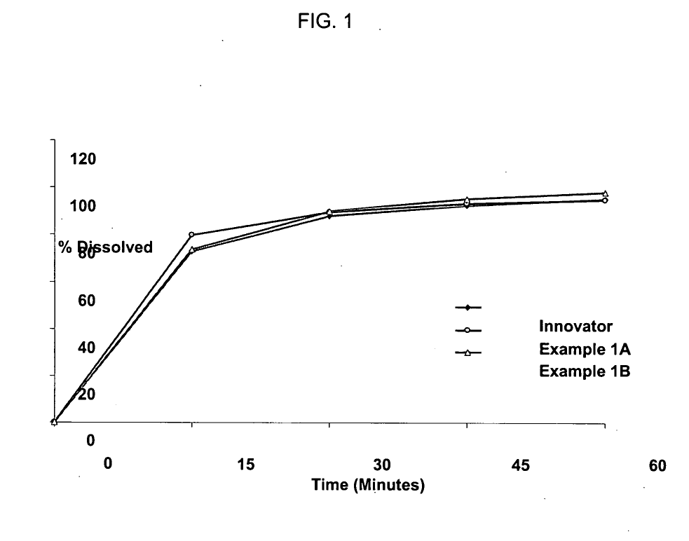

With reference to the drawings, FIG. 1 is a dissolution profile comparison of Modafinil Composition 1A, Composition 1B and Provigil®. The tablets prepared in Example 1, two sets of six tablets each of Composition 1A, Composition 1B and Provigil® were placed in a USP Dissolution Apparatus II set at 50 rpm which contained 900 mL of 0.1 N HCL at 37° C. The percent modafinil dissolved was measured over a period of 60 minutes individually for each of the compositions and Provigil®, and the mean average of the percent modafinil dissolved was plotted.

The results of the dissolution profile set forth in FIG. 1 shows that Compositions 1A and 1B prepared according to the invention exhibit very similar dissolution profiles as compared to Provigil®.

### EXAMPLE 3

### Stability Study Comparison of Modafinil Compositions 1A and 1B Prepared in Example 1, as Compared to Provigil®

A three month stability study was conducted at 40° C. and 75% relative humidity (RH) for 20 tablets individually of Composition 1A, Composition 1B and Provigil®. The percent potency which is an average percent of modafinil determined for each of the samples, and total impurities for each of the samples was determined by HPLC analysis. The results are summarized in Table I.

TABLE I

| Stability | Stability | Potency Assay (%) | | | Total Impurities (%) | | |
|---|---|---|---|---|---|---|---|
| Interval | Station | Provigil ® | 1A | 1B | Provigil ® | 1A | 1B |
| Initial | 25° C./60% RH | 98.3 | | 98.4 | 0.17 | | None |
| 1 Month | 40° C./75% RH | 97.7 | 101.4 | 98.8 | 0.16 | 0.10 | 0.11 |
| 2 Months | 40° C./75% RH | 96.6 | 100.3 | 98.0 | 0.23 | 0.15 | 0.16 |
| 3 Months | 40° C./75% RH | 97.9 | 100.8 | 99.4 | 0.22 | 0.19 | 0.19 |

The results in Table I of the stability study shows that Compositions 1A and 1B prepared according to the invention exhibit a comparable potency assay and total impurities as compared to Provigil® over a three-month period.

### EXAMPLE 4

### Bioequivalency Comparison of Modafinil Compositions 1A and 1B Prepared in Example 1, as Compared to Provigil®

The area under the curve (AUC) and the maximum concentration of modafinil obtained in vivo (Cₘₐₓ) was determined for Modafinil Composition 1A, Composition 1B and Provigil®. The results for Composition 1A and Provigil® are summarized in Table II. The results for Composition 1B and Provigil® are summarized in Table III.

TABLE II

| AUC | | | | Cₘₐₓ | | |
|---|---|---|---|---|---|---|
| | | Composition | Confidence | | Composition | Confidence |
| Study | Provigil ® | 1A | Interval | Provigil ® | 1A | Interval |
| Type | (μg/hr./mL) | (μg/hr./mL) | 90% | (μg/mL) | (μg/mL) | 90% |
| Fasted | 4.054 | 4.060 | 95.6-106 | 1.449 | 1.422 | 90.4-105 |

TABLE III

| AUC | | | | Cₘₐₓ | | |
|---|---|---|---|---|---|---|
| | | Composition | | Composition | | Composition |
| Study | Provigil ® | 1A | Provigil ® | 1A | Provigil ® | 1A |
| Type | (μg/hr./mL) | (μg/hr./mL) | (μg/hr./mL) | (μg/hr./mL) | (μg/hr./mL) | (μg/hr./mL) |
| Fasted | 3.968 | 4.028 | 101-105 | 1.460 | 1.389 | 89.6-96.7 |
| Fed | 4.049 | 4.065 | 99.5-104 | 1.439 | 1.389 | 91.0-99.5 |

The results in Table II and Table III show that the bioequivalency of Compositions 1A and 1B prepared according to the invention exhibit a comparable AUC and Cₘₐₓ as compared to Provigil®.

While the invention has been described with particular reference to certain embodiments thereof, it will be understood that changes and modifications may be made by those of ordinary skill within the scope and spirit of the following claims:

1. A pharmaceutical composition comprising modafinil and at least one silicate in the form of particles, wherein greater than 5% to about 50% of the cumulative total of the modafinil particles in the composition have a particle size greater than about 200 microns as determined by a U.S. Sieve No. 75.

2. The composition according to claim 1 wherein from about 8% to about 30% of the cumulative total of the modafinil particles in the composition have a particle size greater than about 200 microns as determined by a U.S. Sieve No. 75.

3. The composition according to claim 2 wherein from about 8% to about 10% of the cumulative total of the modafinil particles in the composition have a particle size greater than about 200 microns as determined by a U.S. Sieve No. 75.

4. A pharmaceutical composition comprising modafinil and at least one silicate in the form of particles, wherein greater than 5% to about 50% of the cumulative total of the modafinil particles in the composition have a particle size greater than about 200 microns as determined by a U.S. Sieve No. 75, and less than about 8% of the cumulative total of the modafinil particles in the composition have a particle size greater than about 250 microns as determined by a U.S. Sieve No. 60.

5. The composition according to claim 1 wherein less than about 55% of the cumulative total of the modafinil particles in the composition have a particle size greater than about 90 microns as determined by a U.S. Sieve No. 170.

6. (canceled)

7. The composition according to claim 6 claim 1 wherein the silicate is selected from the group consisting of calcium silicate, sodium silicate, magnesium silicate, magnesium trisilicate, and combinations thereof.

8. The composition according to claim 7 wherein the silicate is calcium silicate.

9. The composition according to claim 7 wherein the silicate is a combination of calcium silicate and magnesium trisilicate.

10. (canceled)

11. The composition according to claim 6 claim 1 which is essentially free of magnesium silicate.

12. The composition according to claim 1 wherein modafinil is present in an amount of from about 1 weight percent to about 99 weight percent, based on the total weight of the composition.

13. The composition according to claim 12 wherein modafinil is present in an amount of from about 30 Weight percent to about 50 weight percent, based on the total weight of the composition.

14. The composition according to claim 6 claim 1 wherein the amount of silicate is from about 0.1 weight percent to about 50 weight percent, based on the total weight of the composition.

15. The composition according to claim 14 wherein the amount of silicate is from about 1 weight percent to about 10 weight percent, based on the total weight of the composition.

16. The composition according to claim 15 wherein the amount of silicate is from about 5 weight percent to about 6 weight percent, based on the total weight of the composition.

17. The composition according to claim 1 which additionally comprises one or more excipients.

18. The composition according to claim 17 wherein the excipient is selected from the group consisting of diluents, disintegrants, lubricants, glidants, binders, fillers, emulsifiers, electrolytes, wetting agents, solubilizers, surfactants, colors, pigments, anti-caking agents and combinations thereof.

19. The composition according to claim 18 wherein the diluent is selected from the group consisting of a starch, lactose and microcrystalline cellulose; the disintegrant selected from the group consisting of pre-gelatinized starch, a cross-linked sodium carboxymethyl cellulose, and combinations thereof; and the lubricant is magnesium stearate.

20. A process for preparing a pharmaceutical composition comprising modafinil and at least one silicate, said process comprising: (i) mixing modafinil and at least one silicate to form a mixture; and (ii) optionally mixing other excipients with the mixture formed in Step (i) to form a composition.

21. The process according to claim 20 wherein the composition is in the form of a tablet.

22. The process according to claim 20 wherein the composition is in the form of a capsule.

23. A method of treating a disease or disorder in a subject in need thereof comprising administering to the subject a therapeutically effective amount of the pharmaceutical composition according to claim 1.
